(19) United States
(12) Patent Application Publication
(10) Pub. No.: US 2007/0253863 A1
(43) Pub. Date: Nov. 1, 2007

(54) Method and apparatus for sterilizing a container

(75) Inventors: Takeshi Iwashita, Yokohama-shi (JP); Akira Abe, Tokyo (JP); Chikako Sunohara, Yokohama-shi (JP); Makoto Sawada, Yokohama-shi (JP); Kenichi Kominami, Yokohama-shi (JP)

Correspondence Address: HEDMAN & COSTIGAN P.C., 1185 AVENUE OF THE AMERICAS, NEW YORK, NY, 10036, US

(73) Assignee: TOYO SEIKAN KAISHA, LTD.

(21) Appl. No.: 11/413,588

(22) Filed: Apr. 27, 2006

(51) Int. Cl. A61L 2/18

(52) U.S. Cl. 422/28; 422/302; 422/304

(57) ABSTRACT

A method for sterilizing a container includes a step of spraying a bactericide which has been heated to a temperature within a range from 45° C. to 95° C. over at least an interior surface of the container standing with its mouth being positioned upside in a heated atmosphere. An apparatus for sterilizing a container includes: a temperature regulated sterilizing chamber having an inlet for receiving the container and an outlet for delivering out the container and containing heated air; temperature regulating means for regulating the temperature of the air in the sterilizing chamber to a predetermined temperature; container conveying means for conveying the container standing with its mouth being positioned upside from the inlet to the outlet of the sterilizing chamber; and one or more nozzles provided in the sterilizing chamber for spraying a bactericide which has been heated to a temperature within a range from 45° C. to 95° C. over at least an interior surface of the container conveyed by the container conveying mans.

### BACKGROUND OF THE INVENTION

This invention relates to a method and apparatus for sterilizing containers such as PET bottles filled with drinks or foods such as drinking water, juices, tea and milk coffee.

Known and generally carried out in the art is a method for manufacturing a bottled drink called an aseptic filling method according to which a container such as a PET bottle is sterilized by injecting a bactericide solution such as a mixture solution of hydrogen peroxide, peracetic acid and acetic acid, the sterilized container is rinsed with aseptic rinsing water, a sterilized drink is filled into the sterilized container in an aseptic atmosphere and the container is sealed with a sterilized cap.

As a container sterilizing method used for this aseptic filling method, a sterilizing method is generally employed according to which, as Japanese Patent Application Laid-open Publication No. 2002-332017 discloses in FIG. 2, a container is held in a standing state with its mouth being positioned downside and the container is rinsed by spraying a bactericide solution over both interior and exterior surfaces of the container. Alternatively, Japanese Patent Application Laid-open Publication No. 2001-39414 discloses a container sterilizing method used in the aseptic filling method according to which drops of a bactericide solution are once evaporated and then condensed and mist thus produced is introduced into the container for sterilizing it. Further, alternatively, Japanese Patent Application Laid-open Publication No. Hei 7-291236 discloses a container sterilizing method used in the aseptic filling method according to which a container is sterilized with hot water of 63° C. or over and further sterilized with a bactericide solution.

These prior art methods are disadvantageous in that they involve increased cost for sterilizing containers necessitated by using a large amount of bactericide and that uniform sterilization of the bottom portion of containers cannot be achieved. Therefore, a novel method for sterilizing a container which enables sufficient sterilization of the container and still is capable of reducing the amount of bactericide used.

It is, therefore, an object of the present invention to provide a novel method and apparatus for sterilizing a container which can realize sufficient sterilization of the container while being capable of significantly reducing the amount of use of a bactericide.

### SUMMARY OF THE INVENTION

A method for sterilizing a container for achieving the above described object of the invention comprises a step of spraying a bactericide which has been heated to a temperature within a range from 45° C. to 95° C. over at least an interior surface of the container standing with its mouth being positioned upside in a heated atmosphere.

According to the invention, by spraying a bactericide which has been heated to a temperature within a range from 45° C. to 95° C. over at least an interior surface of a container standing with its mouth being positioned upside in a heated atmosphere, not only the sterilization effect is improved as compared with a case where a bactericide at room temperature is used but also the amount of the bactericide used can be reduced as compared with the prior art method according to which a container is rinsed by injecting a bactericide solution over the container.

Further, when a container is standing with its mouth being positioned downside, it is difficult for a sprayed bactericide to reach the bottom portion of the container and, even if it reaches the bottom portion, since the heated bactericide is readily evaporated, sterilization of the bottom portion cannot be achieved sufficiently within a short period of time and, for achieving sufficient sterilization, spraying time must be prolonged with resulting increase in the amount of the bactericide used. Accordingly to the invention, by spraying a bactericide over an interior surface of a container standing with its mouth being positioned upside, a part of drops of the bactericide deposited on the interior surface of the container flows down to the bottom of the container and stays there while the container is held for a predetermined period of time during the sterilization process whereby sufficient sterilization can be achieved within a relatively short period of time.

More importantly, according to the invention, by conducting spraying of the heated bactericide in a heated atmosphere, when the heated bactericide deposited on the container loses evaporation heat due to evaporation of the heated bactericide with resulting occurrence of tendency toward abrupt drop in the temperature of the deposited bactericide, such abrupt drop in the temperature of the deposited bactericide can be prevented because the container is in the heated atmosphere whereby an expected sterilization effect by the heated bacteicide can be achieved and the amount of use of the bactericide can be held at the minimum.

Further, as the deposited bactericide is evaporated, partial lack of uniformity in the temperature of deposited bactericide tends to occur due to the configuration of the container. Since, however, the container is in the heated atmosphere, such lack of uniformity in the temperature of the deposited bactericide can be held at the minimum and, accordingly, substantially uniform sterilization can be achieved over the entire container surface. In a case where partial lack of uniformity in the temperature of the bactericide exists depending upon the configuration of the container, spraying time of the bactericide must be prolonged for realizing sufficient sterilization over the entire container with resulting increase in the amount of use of the bactericide. According to the invention, substantially uniform sterilization can be conducted over the entire container surface and, in this respect also, the amount of use of the bactericide can be held at the minimum.

In one aspect of the invention, a part of liquid drops of the bactericide deposited on the interior surface of the container stays at the bottom of the container for a predetermined period of time.

In another aspect of the invention, the method comprises a step of detecting the amount of the bactericide staying at the bottom of the container.

An apparatus for sterilizing a container for achieving the above described object of the invention comprises:

a temperature regulated sterilizing chamber having an inlet for receiving the container and an outlet for delivering out the container and containing heated air;

temperature regulating means for regulating the temperature of the air in the sterilizing chamber to a predetermined temperature;

container conveying means for conveying the container standing with its mouth being positioned upside from the inlet to the outlet of the sterilizing chamber; and

one or more nozzles provided in the sterilizing chamber for spraying a bactericide which has been heated to a temperature within a range from 45° C. to 95° C. over at least an interior surface of the container conveyed by the container conveying mans.

In one aspect of the invention, the apparatus further comprises means for inserting the nozzle from the mouth of the container into the container.

In another aspect of the invention, the apparatus further comprises:

a bactericide distribution section for distributing the bactericide;

a bactericide supply path for supplying the bactericide from the bactericide distribution section to the nozzle; and

bactericide heating means for heating the bactericide provided on the bactericide supply path.

According to this aspect of the invention, the bactericide can be heated on the bactericide supply path through which the bactericide passes immediately before being sprayed. A tank storing the bactericide is provided upstream of the bactericide distribution section and the bactericide is supplied to the bactericide distribution section as required. The bactericide may also be heated at a suitable location in a flow path between the bactericide storage tank and the bactericide supply path.

In another aspect of the invention, the temperature regulating means is heating means for supplying and/or circulating heated air to the sterilizing chamber.

In another aspect of the invention, the temperature regulating means is an electric heater capable of heating the air in the sterilizing chamber.

In another aspect of the invention, the temperature regulating means is heating means for supplying and/or circulating a heated fluid through inside of a wall of the sterilizing chamber.

In still another aspect of the invention, the apparatus further comprises a detector provided in the vicinity of the outlet of the sterilizing chamber for detecting the amount of the bactericide staying at the bottom of the container.

### BRIEF DESCRIPTION OF THE DRAWINGS

In the drawings,

FIG. 1 is a plan view schematically illustrating an embodiment of the container sterilizing apparatus according to the present invention;

FIG. 2 is a partial side elevation showing an essential part of the apparatus;

FIG. 3 is a perspective view of the essential part of the apparatus shown with outer walls of the inlet and outlet portions being removed; and

FIG. 4 is a view showing a shower curtain formed by the bactericide solution provided in the vicinity of the inlet and outlet of a temperature regulated sterilizing chamber.

### DESCRIPTION OF PREFERRED EMBODIMENTS

Embodiments of the invention will now be described with reference to the accompanying drawings.

Containers which are the object of sterilization by the present invention include plastic bottles such, for example, as PET bottles and polyolefin bottles and various containers made of plastic or glass as well as metal cans and bottle-shaped cans etc. for containing foods and drinks.

Bactericides used for the container sterilizing method of the present invention typically include bactericide solutions such as peracetic acid bactericide solution, hydrogen peroxide bactericide solution and hypochlorous acid bactericide solution but are not limited to them but any bactericide solution which can sterilize surfaces of a container may be used.

The bactericide used is heated to a temperature within a range from 45° C. to 95° C. The heating temperature is determined within this temperature range having regard to factors such as the type and concentration of the bactericide, type of bacterium which is the object of sterilization and type of contents to be filled in the container. A temperature range which is usually preferable is 64° C. to 70° C.

In the method of the present invention, a container is sterilized by spraying a heated bactericide solution over at least an interior surface of the container standing with its mouth being positioned upside. The reason for spraying the bactericide with the container standing with its mouth being positioned upside is, as described previously, that a part of drops of the bactericide deposited on the interior surface of the container flows down to the bottom of the container and stays there while the container is held for a predetermined period of time during the sterilization process whereby sufficient sterilization can be achieved within a relatively short period of time.

For spraying the heated bactericide over the interior surface of the container, a spray nozzle may be inserted into the container or the spraying may be conducted without the spray nozzle being inserted into the container. An advantage of conducting spraying with the nozzle being inserted into the container is that, in case the container is a bottle, a bactericide spray pattern over the interior surface of the bottle is not obstructed by the diameter of the mouth portion of the bottle. An advantage of conducting spraying without the nozzle being inserted into the container is that the container sterilizing apparatus can be constructed in a simple design and, as a result, the manufacturing cost can be saved.

It is preferable, for achieving sterilization perfectly, that sterilization of a container is conducted not only over an interior surface of the container but simultaneously over an exterior surface of the container.

An important feature of the method of the present invention is that sterilization of a container by spraying of a bactericide is conducted in a heated atmosphere. This may be achieved by, for example, conveying a container through a temperature regulated sterilizing chamber containing heated air and spraying a heated bactericide from a spray nozzle over the container while the container passes through the chamber. The temperature of the heated atmosphere should preferably be about the same or over the heating temperature of the bactericide. Accordingly, the temperature range of the heated atmosphere is, like the heating temperature range of the bactericide, from 45° C. to 95° C. and, more preferably, from 64° C. to 70° C.

By conducting spraying of a bactericide in a heated atmosphere, decrease in the temperature of a deposited bactericide due to loss of evaporation heat can be prevented as described previously and, moreover, decomposition of the bactericide can be reduced. Owing to these advantageous effects, the amount of use of the bactericide can be substantially reduced.

By detecting the amount of the bactericide staying at the bottom of the container in the vicinity of the outlet of the sterilizing chamber, state of spraying of the bactericide can be monitored. By knowing whether a predetermined amount of the bactericide is staying or not, the process management can be made effectively for, for example, ensuring sterilization of the container and checking state of blocking of the nozzle. For detecting the amount of the bactericide staying at the bottom of the container, various means can be used such, for example, as measuring the level of the bactericide solution by means of photo-electric means or image processing, or measuring weight of the container and calculating the amount of the bactericide from the result of the measurement.

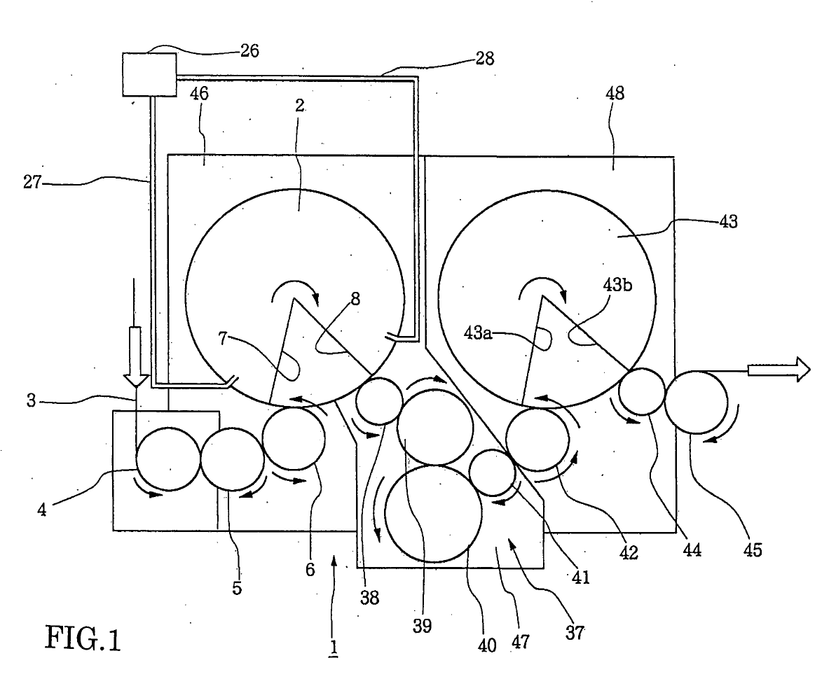

An example of the apparatus for carrying out the method of the present invention will be described with reference to FIGS. 1 to 3.

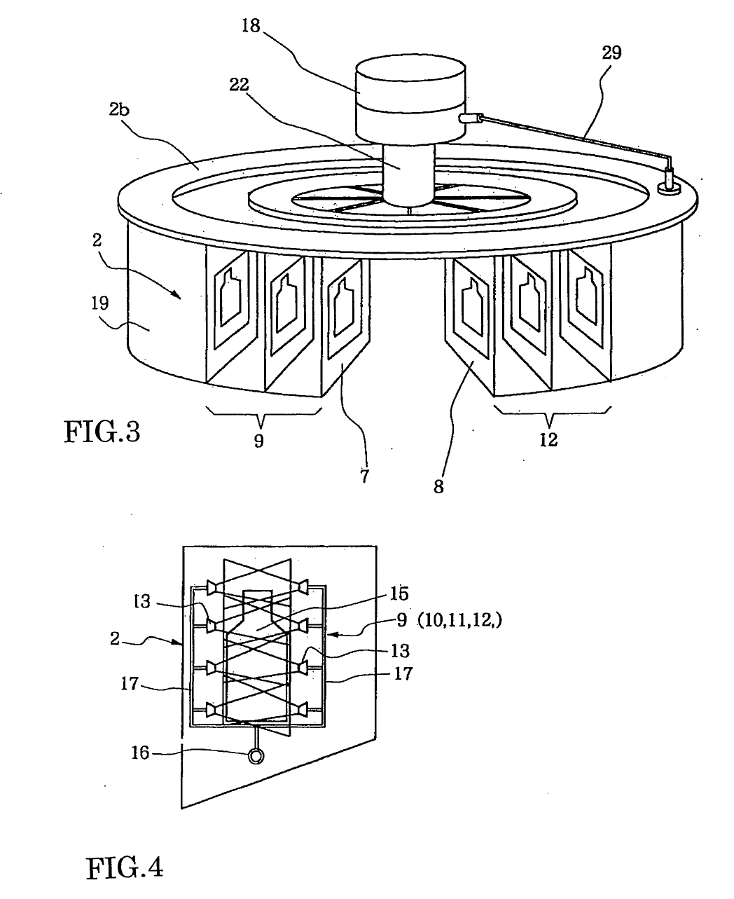

In FIG. 1, a container sterilizing apparatus 1 has a temperature regulated sterilizing chamber 2. A container is conveyed along a container conveyor 3 in the direction of the arrow in standing state with a mouth of the container being positioned upside. The container is conveyed in this state through turrets 4, 5 and 6 to a temperature regulated sterilizing chamber 2 from an inlet 7. The temperature regulated bactericide chamber 2 is constructed, as shown in FIG. 3, in the form of an annular tunnel having the inlet 7 and an outlet 8. In the vicinity of the inlet 7, there are provided shower curtains 9 of a bactericide solution which is regulated in temperature for preventing flowing out of heated air from inside of the sterilizing chamber 2. By these shower curtains 9, flowing of heated air out of the sterilizing chamber 2 is prevented and heating and sterilizing of the exterior surface of the container are performed. Shower curtains 12 of the bactericide solution are also provided in the vicinity of the outlet 8. Although FIG. 3 shows an example in which three shower curtains are provided in each of the inlet 7 and the outlet 8, the number of the shower curtains may be determined suitably having regard to conditions of constructing the container sterilizing line. Each of the shower curtains 9 and 12 is formed, as shown in FIG. 4, by spraying the bactericide solution toward a container 15 from a plurality of nozzles 13 which are provided on both sides of the container in the direction of width of the container 15. Each of the nozzles 13 is connected to a bactericide supply tube 17 which branches off from a bactericide supply main tube 16 to which the bactericide is supplied from a bactericide tank (not shown) storing the bactericide which is provided above the central part of the sterilizing chamber 2.

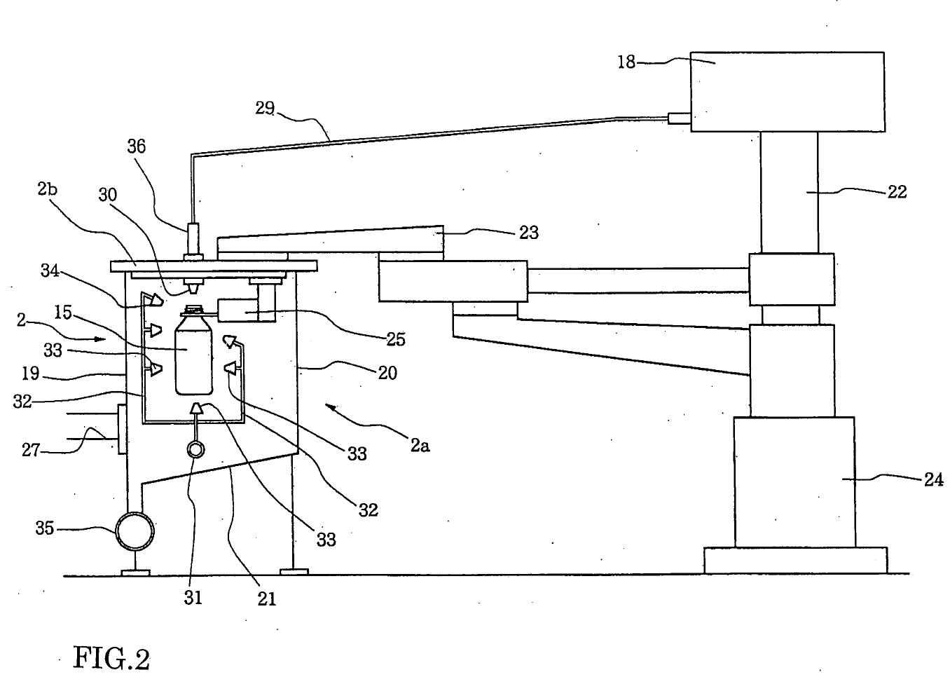

The annular temperature regulated sterilizing chamber 2 is constructed of a stationary section 2a (FIG. 2) comprising an outer peripheral wall 19, an inner peripheral wall 20 and an inclined bottom plate 21, and an annular rotational ceiling plate 2b. The rotational ceiling plate 2b is connected to the foremost end portion of a plurality of ceiling support rods 23 which extend from and fixed to a central rotation shaft 22 disposed vertically in the center of the sterilizing chamber 2. As the rotation shaft 22 is rotated by an electric motor 24 to which the rotation shaft 22 is connected, the rotational ceiling plate 2b is rotated along the upper periphery of the stationary section 2a. For keeping heat, the outer peripheral wall 19, the inner peripheral wall 20 and the bottom plate 21 should preferably be constructed in dual plate construction.

In this example, the ceiling plate 2b rotates but, alternatively, by changing disposition of a spray nozzle 30 and a container holder 25 to be described below, the ceiling plate may be made stationary and the inner peripheral wall may be rotated.

Container holders 25 are fixed at a predetermined interval on the lower surface of the rotational ceiling plate 2b. As the container 15 is conveyed to the inlet 7 of the sterilizing chamber 2, the container 15 is held by the container holder 25 and is conveyed through the sterilizing chamber 2 to the outlet 8. The rotational ceiling plate 2b therefore constitutes the container conveying means in the present invention.

For stabilizing the temperature in the temperature regulated sterilizing chamber 2, heated air is supplied to the sterilizing chamber 2. An end portion of a heated air supply duct 27 connected to a heated air supply source 26 (FIG. 1) is opened in the outer peripheral wall 19 of the sterilizing chamber 2 in the vicinity of the inlet 7 and end portion of a heated air exhaust duct 28 is opened in the outer peripheral wall 19 in the vicinity of the outlet 8. Air which has been heated to a temperature which is about the same or over temperature of the bactericide is supplied from the heated air supply source 26 to the sterilizing chamber 2 through the heated air supply duct 27. The heated air returns to the heated air supply source 26 through the heated air exhaust duct 28. Therefore, the temperature of inside atmosphere of the temperature regulated sterilizing chamber 2 is maintained at about the same or over the temperature of the heated bactericide. For keeping the temperature of the sterilizing chamber 2, a suitable system such as a system of using insulating material in the walls of the sterilizing chamber 2, a system of providing a heating equipment such as an electric heater in the sterilizing chamber which can control the temperature of the atmosphere in the sterilizing chamber, a system of supplying and/or circulating heated fluid or steam through a tube provided in the walls of the sterilizing chamber or in the sterilizing chamber or a combination of such systems may be used.

A bactericide distribution section 18 is fixed to the upper end portion of the rotation shaft 22. To the bactericide distribution section 18 are connected a plurality of bactericide supply tubes 29 at a predetermined interval and at positions corresponding to the fixed positions of the container holders 25. The bactericide supply tubes 29 constitute the bactericide supply paths of the invention. Although a plurality of bactericide supply tubes 29 are provided, only one bactericide supply tube 29 is illustrated in FIG. 3 for convenience of illustration. A spray nozzle 30 (FIG. 2) for sterilizing the interior surface of the container 15 is provided facing the mouth of the container 15 on the interior surface of the rotational ceiling plate 2b at a position above the mouth of the container 15. This nozzle 30 is connected to each of the bactericide supply tubes 29. A tube-shaped heater 36 is provided in the end portion of the bactericide supply tube 29 in the vicinity of the nozzle 30 for heating the bactericide to a desired temperature within a range from 45° C. to 95° C. Therefore, the spray nozzles 30 are rotated with the rotational ceiling plate 2b to continuously spray the heated bactericide over the interior surfaces of the containers 15. The tube-shaped heater 36 constitutes the bactericide heating means provided on the bactericide supply path.

In the stationary section 2a of the regulated temperature sterilizing chamber 2 are provided, at a predetermined interval, a plurality of stationary spray nozzles 33 on both sides of the container 15 and below the bottom of the container 15 so as to spray the bactericide over the exterior surface and the outer bottom surface of the passing container 15. These spray nozzles 33 are connected to bactericide supply branch tubes 32 branching from a bactericide supply tube 31. A tube-shaped heater (not shown) is provided on the bactericide supply tube 31 to heat the bactericide to a predetermined temperature.

A rinsing nozzle 34 is provided for injecting rinsing water over the container holder 25 to rinse the container holder 25. There is also provided a drain pipe 35 for draining the bactericide which has completed sterilization of the container 15.

Referring again to FIG. 1, adjacent to the outlet 8 of the sterilizing chamber 2 is provided a container reversing and bactericide draining section 37 consisting of turrets 38, 39, 40 and 41. A container standing with its mouth being positioned upside which has completed sterilization and has come out of the outlet 8 is turned upside down while it is conveyed by rotation of the turrets 38, 39, 40 and 41 of the container reversing and bactericide draining section 37 and bactericide which has remained in the container is thereby drained. An arrangement may be made so as to temporarily hold the bactericide at the mouth portion of the container in draining the bactericide from the container.

The container which has been turned upside down with the bactericide having been drained is conveyed from the turret 41 through a turret 42 to the inside of a rinser 43 from its inlet 43a. As the rinser 43, a known rinser which rinses a container by spraying or injecting aseptic rinsing water from a nozzle (not shown) over at least an interior surface, and preferably over both interior and exterior surfaces, of the container which stands with its mouth being positioned downside may be used.

The container which has been rinsed by the rinser 43 is conveyed through turrets 44 and 45 to subsequent processes such as filling of a drink in the container.

In the above described embodiment, shower curtains formed by spraying a bactericide are employed for preventing flowing out of heated air out of the regulated temperature sterilizing chamber 2. Alternatively, other heat insulating means such, for example, as heat insulating means using heated wind may be employed. Further, there may be provided, in a stage prior to the inlet 7 of the sterilizing chamber 2, a preliminary container heating section which heats a container by means of, for example, a device which replaces air in the container by heated air.

### EXAMPLE

A sterilization effect was tested by using a 500 ml PET bottle as a container and using a peracetic acid bactericide Toyo-aktiv (TM) with peracetic acid concentration of 2,000 ppm as a bactericide. This PET bottle was held in a temperature regulated chamber in a state standing with its mouth being positioned upside in a heated atmosphere under a temperature within a range from 60° C. to 64° C. and the bactericide heated to 67° C. was sprayed over the interior surface of the PET bottle. As the spray nozzle, a full corn type nozzle was used and the PET bottle was disposed at a position at which the tip of the nozzle was positioned 20 mm above the mouth of the PET bottle so that a spray pattern of the bactericide sprayed from the nozzle will reach the interior surface of the PET bottle. The flow rate of the bactericide was 0.6 L/min at 0.2 MPa.

As a bacterium to be tested, Bacillus subtilis var. glogigii IFO (NBRC) 13721 was used and sterilization of the PET bottle by spraying the bactericide was conducted. After sterilization for 4 seconds, 8 seconds and 12 seconds, the number of bacteria surviving on the interior surface of the PET bottle was respectively measured. Results are shown in Table 1. The number of surviving bacteria at each sterilization time is results of tests of n=3 and average of the results.

TABLE 1
Bacilus subtilis var. glogigii IFO (NBRC) 13721
Sterilization Test (n = 3)
Initial number of bacteria (cfu/bottle) 4.8 × 10⁶

| Steriliza- | | | | | Steriliza- |
|---|---|---|---|---|---|
| tion time | Number of surviving bac. (cfu/bottle) | | | | tion effect |
| (seconds) | \textcircled{1} | \textcircled{2} | \textcircled{3} | average | (D) |
| 4 | 0 | 0 | 0 | 0 | >7.2 |
| 8 | 0 | 0 | 0 | 0 | >7.2 |
| 12 | 0 | 0 | 0 | 0 | >7.2 |

As a result of the sterilization test, it has been found that the amount of use of the bactericide according to the prior art sterilization method in which a container standing with its mouth being positioned downside is rinsed with the bactericide is about 6 litters per minute whereas, according to the method of the present invention, the amount of use of the bactericide for achieving the same sterilization effect can be reduced to about 0.6 litter per minute.

What is claimed is:

1. A method for sterilizing a container comprising a step of spraying a bactericide which has been heated to a temperature within a range from 45° C. to 95° C. over at least an interior surface of the container standing with its mouth being positioned upside in a heated atmosphere.

2. A method as defined in claim 1 wherein a part of liquid drops of the bactericide deposited on the interior surface of the container stays at the bottom of the container for a predetermined period of time.

3. A method as defined in claim 2 comprising a step of detecting the amount of the bactericide staying at the bottom of the container.

4. An apparatus for sterilizing a container comprising:
a temperature regulated sterilizing chamber having an inlet for receiving the container and an outlet for delivering out the container and containing heated air;
temperature regulating means for regulating the temperature of the air in the sterilizing chamber to a predetermined temperature;
container conveying means for conveying the container standing with its mouth being positioned upside from the inlet to the outlet of the sterilizing chamber; and
one or more nozzles provided in the sterilizing chamber for spraying a bactericide which has been heated to a temperature within a range from 45° C. to 95° C. over at least an interior surface of the container conveyed by the container conveying means.

5. An apparatus as defined in claim 4 further comprising means for inserting the nozzle from the mouth of the container into the container.

6. An apparatus as defined in claim 4 further comprising:
a bactericide distribution section for distributing the bactericide;
a bactericide supply path for supplying the bactericide from the bactericide distribution section to the nozzle; and
bactericide heating means for heating the bactericide provided on the bactericide supply path.

7. An apparatus as defined in claim 5 further comprising:
a bactericide distribution section for distributing the bactericide;
a bactericide supply path for supplying the bactericide from the bactericide distribution section to the nozzle; and
bactericide heating means for heating the bactericide provided on the bactericide supply path.

8. An apparatus as defined in claim 4 wherein the temperature regulating means is heating means for supplying and/or circulating heated air to the sterilizing chamber.

9. An apparatus as defined in claim 4 wherein the temperature regulating means is an electric heater capable of heating the air in the sterilizing chamber.

10. An apparatus as defined in claim 4 wherein the temperature regulating means is heating means for supplying and/or circulating a heated fluid through inside of a wall of the sterilizing chamber.

11. An apparatus as defined in any of claims 4-10 further comprising a detector provided in the vicinity of the outlet of the sterilizing chamber for detecting the amount of the bactericide staying at the bottom of the container.
